(19) United States
(12) Patent Application Publication
(10) Pub. No.: US 2003/0031660 A1
(43) Pub. Date: Feb. 13, 2003

(54) Method of inducing bone formation

(75) Inventor: Oron Yacoby-Zeevi, Moshav Bizaron (IL)

Correspondence Address: G.E. EHRLICH (1995) LTD., c/o ANTHONY CASTORINA, SUITE 207, 2001 JEFFERSON DAVIS HIGHWAY, ARLINGTON, VA, 22202, US

(73) Assignee: Insight Strategy & Marketing Ltd.

(21) Appl. No.: 10/163,993

(22) Filed: Jun. 7, 2002

Related U.S. Application Data
(63) Continuation of application No. 09/260,037, filed on Mar. 2, 1999.
Application No. 09/260,037 is a continuation-in-part of application No. 09/140,888, filed on Aug. 27, 1998, now Pat. No. 6,423,312.
Application No. 09/140,888 is a continuation-in-part of application No. 09/046,475, filed on Mar. 25, 1998, now Pat. No. 6,153,187.
Application No. 09/046,475 is a continuation-in-part of application No. 08/922,170, filed on Sep. 2, 1997, now Pat. No. 5,968,822.

(51) Int. Cl. A61K 38/47

(52) U.S. Cl. 424/94.61

(57) ABSTRACT

A method of inducing bone formation in a subject in need thereof, the method comprises administering to the subject a therapeutically effective amount of heparanase. A pharmaceutical composition for inducing bone formation in a subject in need thereof, the pharmaceutical composition comprises, as an active ingredient, a therapeutically effective amount of heparanase, and a pharmaceutically acceptable carrier.

This is a Continuation of U.S. patent application Ser. No. 09/260,037, filed Mar. 2, 1999, which is a Continuation-In-Part of U.S. patent application Ser. No. 09/140,888, filed Aug. 27, 1998, which is a Continuation-In-Part of U.S. patent application Ser. No. 09/046,475, filed Mar. 25, 1998, now U.S. Pat. No. 6,153,187, issued Nov. 28, 2000, which is a Continuation-In-Part of U.S. patent application Ser. No. 08/922,170, filed Sep. 2, 1997, now U.S. Pat. No. 5,968,822, issued Oct. 19, 1999, the specifications thereof are incorporated herein by reference.

### FIELD AND BACKGROUND OF THE INVENTION

The present invention relates to methods, preparations and pharmaceutical compositions for introducing biological materials into patients. In particular, the present invention related to methods, preparations and pharmaceutical compositions for efficiently introducing cells, tissues and drug delivery systems into patients. The present invention further relates to a method and pharmaceutical composition for inducing bone formation.

Proteoglycans (PGs): Proteoglycans (previously named mucopolysaccharides) are remarkably complex molecules found in every tissue of the body. PGs are associated with each other and also with the other major structural components of cells and tissues, such as collagen and elastin. Some PGs interact with certain adhesive proteins, such as fibronectin and laminin. The long extended nature of the polysaccharide chains of glycosaminoglycans (GAGs) and their ability to gel, allow relatively free diffusion of small molecules, but restrict the passage of large macromolecules. Because of their extended structures and the huge macromolecular aggregates they often form, PGs occupy a large volume of the extracellular matrix relative to proteins [Murry R K and Keeley F W; Biochemistry, Ch. 57. pp. 667-85].

Heparan sulfate proteoglycans (HSPG) are acidic polysaccharide-protein conjugates associated with cell membranes and extracellular matrices. They bind avidly to a variety of biologic effector molecules, including extracellular matrix components, growth factors, growth factor binding proteins, cytokines, cell adhesion molecules, proteins of lipid metabolism, degrading enzymes, and protease inhibitors. Owing to these interactions, heparan sulfate proteoglycans play a dynamic role in biology, in fact most functions of the proteoglycans are attributable to the heparan sulfate chains, contributing to cell-cell interactions and cell growth and differentiation in a number of systems. Heparan sulfate maintains tissue integrity and endothelial cell function. It serves as an adhesion molecule and presents adhesion-inducing cytokines (especially chemokines), facilitating localization and activation of leukocytes. Heparan sulfate modulates the activation and the action of enzymes secreted by inflammatory cells. The function of heparan sulfate changes during the course of the immune response are due to changes in the metabolism of heparan sulfate and to the differential expression of, and competition between, heparan sulfate-binding molecules [Selvan R S et al., Ann. N Y Acad. Sci. 1996, 797: 127-39].

HSPGs are also prominent components of blood vessels [Wight T N et al., Arteriosclerosis, 1989, 9: 1-20]. In large vessels they are concentrated mostly in the intima and inner media, whereas in capillaries they are found mainly in the subendothelial basement membrane where they support proliferating and migrating endothelial cells and stabilize the structure of the capillary wall. The ability of HSPGs to interact with extracellular matrix (ECM) macromolecules such as collagen, laminin and fibronectin, and with different attachment sites on plasma membranes suggests a key role for this proteoglycan in the self-assembly and insolubility of ECM components, as well as in cell adhesion and locomotion.

Heparanase—a GAGs degrading enzyme: Degradation of GAGs is carried out by a battery of lysosomal hydrolases. One important enzyme involved in the catabolism of certain GAGs is heparanase. It is an endo-β-glucuronidase that cleaves heparan sulfate at specific interchain sites. Interaction of T and B lymphocytes, platelets, granulocytes, macrophages and mast cells with the subendothelial extracellular matrix (ECM) is associated with degradation of heparan sulfate by heparanase activity. Connective tissue activating peptide III (CTAP), an α-chemokine, can act as a heparanase, and some heparanases act as adhesion molecules or as degrading enzymes depending on the pH of the micro microenvironment. The enzyme is released from intracellular compartments (e.g., lysosomes, specific granules) in response to various activation signals (e.g., thrombin, calcium ionophore, immune complexes, antigens and mitogens), suggesting its regulated involvement in inflammation and cellular immunity [Vlodavsky I et al., Invasion Metas. 1992; 12(2): 112-27]. In contrast, various tumor cells appear to express and secrete heparanase in a constitutive manner in correlation with their metastatic potential [Nakajima M et al., J. Cell. Biochem. 1988 Feb; 36(2): 157-67].

Important processes in the process of tissue invasion by leukocytes include their adhesion to the luminal surface of the vascular endothelium, their passage through the vascular endothelial cell layer and the subsequent degradation of the underlying basal lamina and extracellular matrix with a battery of secreted and/or cell surface protease and glycosidase activities. Cleavage of heparan sulfate by heparanase may therefore result in disassembly of the subendothelial ECM and hence may play a decisive role in extravasation of normal and malignant blood-borne cells [Vlodavsky I et al., Inv. Metast. 1992, 12: 112-27, Vlodavsky I et al., Inv. Metast. 1995, 14: 290-302].

It has been previously demonstrated that heparanase may not only function in cell migration and invasion, but may also elicit an indirect neovascular response [Vlodavsky I et al., Trends Biochem. Sci. 1991, 16: 268-71]. The ECM HSPGs provide a natural storage depot for basic fibroblast growth factor (bFGF). Heparanase mediated release of active bFGF from its storage within ECM may therefore provide a novel mechanism for induction of neovascularization in normal and pathological situations [Vlodavsky I et al., Cell. Molec. Aspects. 1993, Acad. Press. Inc. pp. 327-343, Thunberg L et al., FEBS Lett. 1980, 117: 203-6]. Degradation of heparan sulfate by heparanase results in the release of other heparin-binding growth factors, as well as enzymes and plasma proteins that are sequestered by heparan sulfate in basement membranes, extracellular matrices and cell surfaces [Selvan R S et al., Ann. N Y Acad. Sci. 1996, 797: 127-39].

The use of marrow stromal cells for cell and gene therapy: Bone arrow stromal cells (MSCs) have the potential to differentiate into a variety of mesenchymal cells. Within the past several years MSCs have been explored as vehicles for both cell and gene therapy. These cells are relatively easy to isolate from small aspirates of bone marrow that can be obtained under local anesthesia; they are also relatively easy to expand in culture and to transfect with exogenous genes. Several different strategies are being pursued for the therapeutic use of MSCs as follows:

(i) Isolation of MSCs from the bone marrow of a patient with degenerative arthritis, expansion of the MSCs in culture, and then use the expanded cells for resurfacing of joint surfaces by direct injection into the joints. Alternatively, the MSCs can be implanted into a poorly healing bone to enhance the repair process thereof.

(ii) Introduction of genes encoding secreted therapeutic proteins into the MSCs and then infuse the cells systemically so that they return to the marrow or other tissues and secrete the therapeutic protein. Infused MSCs systemically, under conditions in which the cells not only repopulate bone marrow, also provide progeny for the repopulation of other tissues such as bone, lung and perhaps cartilage and brain. Recent experiments showed that when donor MSCs from normal mice are infused in large amounts into young mice that are enfeebled because they express a mutated collagen gene, the normal donor cells replace up to 30% of the cells in bone, cartilage, and brain of the recipient mice. These results were the basis of a clinical trial now in progress for the therapy of bone defects seen in children with sever osteogenesis imperfecta caused by mutations in the genes for type I collagen [Prockop D J; Science 1997, 276: 71-74]. Treatment and potential cure of lysosomal and peroxisomal diseases, heretofore considered fatal, has become a reality during the past decade. Bone marrow transplantation, has provided a method for replacement of the disease-causing enzyme deficiency. Cells derived form the donor marrow continue to provide enzyme indefinitely. Several scores of patients with diseases as diverse as metachromatic leukodystrophy, adrenoleukodystrophy, Hurler syndrome (MPS I), Maroteaux-Lamy (MPS VI), Gaucher disease, and fucosidosis have been successfully treated following long term engraftment. Central nervous system (CNS) manifestations are also prevented or ameliorated in animal models of these diseases following engraftment from normal donors. The microglial cell system has been considered to be the most likely vehicle for enzyme activity following bone marrow engraftment. Microglia in the mature animal or human are derived form the newly engrafted bone marrow [Krivit W et al., Cell Trans. 1995, 4(4): 385-92]. In animal models, MSCs can be transfected using retroviruses and can achieve high-level gene expression both in vitro and in vivo [Lazarus H M et al., Bone Marrow Transpl. 1995, 16, 557-64].

(iii) MSCs secreting a therapeutic protein can be encapsulated in some inert material that allows diffusion of proteins but not of the cells themselves. It was shown that human MSCs transfected with a gene for factor IX secrete the protein for at least 8 weeks after systemic infusion into SCID mice [Prockop D J; Science 1997, 276: 71-74].

The pluripotential nature of marrow stromal fibroblasts (MSFs) is well documented. However, factors that stimulate their initial proliferation and subsequent maturation are not well established. Only bFGF was found to slightly stimulate proliferation [Gehron Robey P et al., 6^{th} international conference on the molecular biology and pathology of matrix, session IV]. Others have demonstrated the marked difficulty in transplanting stromal cells to the bone marrow; stromal cells transplanted into immunodeficient mice may survive in spleen, liver, or lung but not in bone marrow [Lazarus H M et al., Bone Marrow Transpl. 1995, 16, 557-64].

The use of primary skin fibroblasts and keratinocytes for cell and gene therapy: The skin plays a crucial role in protecting the integrity of the body's internal milieu. The loss of substantial portions of this largest organ of the body is incompatible with sustained life. In reconstructive surgery or burn management, substitution of the skin is often necessary. In addition to traditional approaches such as split or full thickness skin grafts, tissue flaps and free-tissue transfers, skin bioengineering in vitro or in vivo has been developing over the past decades [Pomahac B et al, Crit Rev Oral Biol Med 1998, 9(3): 333-44].

Flap prefabrication is dependent on the neovascular response that occurs between the implanted arteriovenous pedicle and the recipient tissue. Augmentation of this neovascular response with angiogenic growth factors would maximize flap survival and minimize the interval between pedicle implantation and flap rotation. Maximizing the biological activity of endogenous growth factors would likewise positively impact upon flap survival. The use of substrates designed to maximize the biological activity of endogenous growth factors, rather than relying on the artificial addition of exogenous growth factors, represents a new approach in the search for methods that will improve flap survival [Duffy F J Jr et al., Microsurg. 1996, 17(4): 176-9].

Clinical strategies to decrease hypertrophic scar should include an attempt at early wound closure with skin grafting or the application of cultured epithelial autografts [Garner W L, Plast Reconstr Surg 1998, 102(1): 135-9].

Epidermal and dermal cells can be multiplied in vitro using different techniques. Autologous epidermal substitutes for wound coverage in deep bums are prepared in less than three weeks. New technologies are required to optimize the nutrition of 3-dimensional cultures of skin cells, which should lead to further progress in the area of skin reconstruction [Benathan M et al., Rev Med Suisse Romande 1998, 118(2): 149-53].

Cultured epithelial autografts offer an exciting approach to cover extensive skin wounds. The main problem of this method is mechanical instability during the first weeks after grafting. There is evidence that the shortcomings of autografting cultured keratinoncytes result from the lack of a mature and functional dermo-epidermal junction [Raghunath M et al., Pediatr Surg Int 1997, 12(7): 478-83].

Keratinocyte grafting can be used to treat acute traumatic and chronic non-healing wounds. The keratinocyte sheets are fragile and clinical take is difficult to assess, especially as activated keratinocytes secrete many growth factors, which have effects on wound healing apart from take. There is now overwhelming evidence of the requirement for a dermal substitute for cultured keratinocyte autografts [Myers S et al., Am J Surg 1995, 170(1): 75-83].

Genetic modification of primary skin fibroblasts offers a new approach to the focal delivery of deficient transmitter-specific enzymes or trophic substances to the damaged or diseased CNS. Although fibroblasts are unable to provide anatomical corrections to defective neural connectivity, they can serve as biological pumps for the enzymes and growth factors in vivo. The capability of genetically engineered cells to ameliorate disease phenotypes in animal models of CNS disorders may ultimately result in the restoration of function. At this time, primary skin fibroblasts appear to be a convenient cellular population for the application of gene transfer and intracerebral grafting for the animal model of Parkinson's disease [Kawaja M D et al., Genet Eng (NY) 1991, 13: 205-20].

The use of enzymes for gene delivery: The use of ECM-degrading enzymes for cell or gene therapy is very limited. One report showed that pre-incubation with elastase increased the transduction efficiency of catheter-based gene delivery of replication-defective adenoviral vectors to rabbit iliac arteries without detectable arterial damage. The major barrier to percuatneous adenovirus mediated gene delivery to the arterial media appears to be the internal elastic lamina [Maillard L et al., Gene therapy 1998, 5, 1023-30].

The role of ECM and bFGF in tissue regeneration: The ECM HSPGs provide a natural storage depot for basic fibroblast growth factor (bFGF). Heparanase mediated release of active bFGF from its storage within ECM may therefore provide a novel mechanism for induction of neovascularization in normal and pathological situations [Vlodavsky I et al., Cell. Molec. Aspects. 1993, Acad. Press. Inc. pp. 327-343, Thunberg L et al., FEBS Lett. 1980, 117: 203-6]. bFGF is one of the endogenous factors found in bone matrix. bFGF is a mitogen for many cell types, including osteoblasts and chondrocytes. A lower dose of bFGF increases bone calcium content and a higher dose reduces it. Thus, exogenous bFGF can stimulate proliferation during early phases of bone induction. bFGF stimulates bone formation in bone implants, depending on dose and method for administration. Hyaluronate gel has been shown to be effective as a slow-release carrier for bFGF [Wang J S, Acta Orthop. Scand. Suppl. 1996, 269: 1-33]. bFGF infusion increases bone ingrowth into bone grafts when infused at both an early and a later stage, but the effect can be measured only several weeks later [Wang J S et al., Acta Orthop Scand 1996, 67(3): 229-36].

bFGF has been reported to increase the volume of callus in a fracture model of rats. There are, however, no reports of successful repair of segmental bony defects by application of an bFGF solution. An adequate dose of bFGF and an appropriate delivery system are required for successful healing of large bony defects. These findings imply the potential value of bFGF minipellets in clinical practice [Inui K et al., Calcif Tissue Int 1998, 63(6): 490-5].

Bone regeneration by bFGF complexed with biodegradable hydrogels was used for repair of skull bone defects which has been clinically recognized as almost impossible [Tabata Y et al., Biomaterials 1998, 19(7-9): 807-15].

Implantation of demineralized bone matrix in rodents elicits a series of cellular events leading to the formation of new bone inside and adjacent to the implant. This process was believed to be initiated by an inductive protein present in bone matrix. It has been suggested that local growth factors may further regulate the process once it has been initiated. Bone formation was induced by all the implants after 3 weeks. The amount of mineralized tissue in the bFGF-treated implants was 25 percent greater than in untreated controls [Aspenberg P et al., Acta Orthop Acand 1989, 60(4): 473-6].

Local application of recombinant human bFGF in a carboxymethyl cellulose gel to demineralized bone matrix implants increases the bone yield as measured by calcium content 3 weeks after implantation in rats. This increase was seen at 3 and 4 weeks, but not earlier or later. Furthermore, the stimulatory effect was seen with doses from 3 to 75 ng per implant. A dose of 0.6 or 380 ng did not increase the bone yield and 1900 ng had a marked inhibitory effect [Aspenberg P et al., Acta Orthop Acand 1991, 62(5): 481-4].

Omental implantation, a surgical procedure in which a perforated gastric or duodenal ulcer is repaired by drawing and implanting a portion of the omentum into the digestive tract, accelerates ulcer healing and inhibits ulcer recurrence. Greater anti-inflammatory and angiogenic activity and accelerated collagen synthesis were seen in the omental implantation group. bFGF-mediated angiogenesis was noted in this group, as well as TGF-β1 activity within and around the omentum [Matoba Y et al., J. Gastroenterol. 1996, 31(6): 777-84].

Application of bFGF restored the formation in healing-impaired rat models treated with steroid, chemotherapy and X-ray irradiation. Repeated applications of bFGF accelerated closure of full-thickness excisional wounds in diabetic mice, but the high doses showed rather diminished responses. In contrast, histological and gross evaluation of wound tissues revealed enhanced angiogenesis and granulation tissue formation in a dose-dependent manner. These findings suggest that the topical application of excess amounts of bFGF might reduce its ability to promote wound closure because of the prolonged responses in both neovascular and granulation tissue formation [Okumura M et al., Arzneimittelforschung 1996, 46(10): 1021-6].

The levels of endogenous bFGF in control and ischemic hind limbs, and the response to the administration of exogenous recombinant bFGF and heparin were documented. Following arterial occlusion there was a ten-fold increase in the levels of endogenous bFGF in all ischemic muscle groups. Intramuscular implantation of bFGF in heparin-sepharose pellets at the time of arterial ligation markedly enhanced the blood flow for 3 weeks compared with untreated ischemic limbs. A further increment in blood flow occurred if an additional dose of bFGF was administered 4 weeks after ligation [Chleboun J O and Martins R N; Aust. N Z J Surg. 1994, 64(3): 202-7].

The involvement of ECM and bFGF in blastocyst implantation: At implantation, trophectoderm attaches to the apical uterine luminal epithelial cell surface. Molecular anatomy studies in humans and mice, and data from experimental models have identified several adhesion molecules that could take part in this process: integrins of the alpha v family, trophinin, CD44, cad-11, the H type I and Lewis y oligosaccharides and heparan sulfate. After attachment, interstitial trophoblast invasion occurs requiring a new repertoire of adhesive interactions with maternal ECM as well as stromal and vascular cell populations. Human anchorage sites contain columns of cytotrophoblasts in which self-attachment gives way progressively to adhesion to ECM and then interstitial migration [Aplin J D; Rev Reprod 1997, 2(2): 84-93. Lessey BA et al., J Reprod Immuol 1998, 39(1-2): 105-16].

During the process of implantation in humans, fetal trophoblast cells invade and migrate into the maternal decidua. During this migration, trophoblast cells destroy the wall of the maternal spiral arteries, converting them from muscular vessels into flaccid sinusoidal sacs. This vascular transformation is important to ensure an adequate blood supply to the feto-placental unit. Both cell-cell and cell-matrix interactions are important for trophoblast invasion of the decidual stroma and decidual spiral arteries. Cell-matrix adhesions are mediated by specific receptors, mostly belonging to the family of integrins. Signals transduced to the cells from the matrix via integrins could play a pivotal role in the control of cellular behavior and gene expression, such as metalloproteinases that facilitate matrix degradation and tissue remodeling [Burrows T D et al., Hum Reprod Updat 1996, 2(4): 307-21]. Thus, the trophoblastic cells of the blastocyst and of the placenta express an invasive phenotype. These cells produce and secrete metalloproteinases which are capable of digesting the extracellular matrix and invade it. Among the numerous endometrial factors that control trophoblastic invasion, the components of the ECM such as laminin and fibronectin, play an important role. The endometrial extracellular matrix is thus a potent regulator of trophoblast invasion [Bischof P et al., Contracept Fertil Sex 1994, 22(1): 48-52]. The invasion of extravillous trophoblast cells into the maternal endometrium is one of the key events in human placentation. The ability of these cells to infiltrate the uterine wall and to anchor the placenta to it, as well as their ability to infiltrate and to adjust utero-placental vessels to pregnancy depends, among other things, reflect on their ability to secrete enzymes that degrade the extracellular matrix [Huppertz B et al., Cell Tissue Res. 1998, 291(1): 133-48].

Expression of the heparan sulfate proteoglycan, perlecan, on the external trophectdermal cell surfaces of mouse blastocysts increases during acquisition of attachment competence [Smith S E et al., Dev. Biol. 1997, 184(1): 38-47]. Radioautographic data indicates that mouse decidual cells produce and secrete collagen and sulfated proteoglycans [Abrahamsohn P A et al., J. Exp. Zool. 1993 266(6): 603-28].

Heparan sulfate proteoglycan (HSPG) is an integral constituent of the placental and decidual ECM. Because this proteoglycan specifically interacts with various macromolecules in the ECM, its degradation may disassemble the matrix. Hence, in the case of the placenta, this may facilitate normal placentation and trophoblast invasion. Incubation of cytotrophoblasts in contact with ECM results in release of ECM-bound bFGF. It has been, therefore, proposed that the cytotrophoblastic heparanase facilitates placentation, through cytotrophoblast extravasation and localized neovascularization [Goshen R et al., Mol. Hum. Reprod. 1996, 2(9): 679-84].

Mammalian embryo implantation involves a series of complex interactions between maternal and embryonic cells. Uterine polypeptide growth factors may play critical roles in these cell interactions. bFGF is a member of a family of growth factors. This growth factor may be potentially important for the process of embryo implantation because (i) it is stored within the ECM and is thus easily available during embryo invasion; (ii) it is a potent modulator of cell proliferation and differentiation; and (iii) it stimulates angiogenesis [Chai N et al., Dev. Biol. 1998, 198(1): 105-15]. Relatively high concentrations of bFGF significantly enhance the rates of blastocyst attachment and of trophoblast spreading and promote the expansion of the surface area of the implanting embryos. Keratinocyte growth factor (KGF) and bFGF derived form the endometrial cells exert paracrine effects on the process of implantation by stimulating trophoblast outgrowth through their cognate receptors [Taniguchi F et al., Mol. Reprod. Dev. 1998, 50(1): 54-62; Yoshida S; Nippon Sanka Fujinka Gaddai Zasshi 1996, 48(3): 170-6].

The mRNAs encoding bFGF were detected in all stages of preimplantation embryos, although the relative abundance of this transcript decreased from the single cell to the blastocyst stage, suggesting that it may represent a maternal transcript in early sheep embryos. The expression of growth factor transcripts very early in mammalian development would predict that these molecules fulfill necessary role(s) in supporting the progression of early embryos through the preimplantation interval [Watson A J et al., Biol Reprod. 1994, 50(4): 725-33].

The cellular distribution of bFGF was examined immunohistochemically in the rat uterus during early pregnancy (days 2-6). bFGF localized intracellularly in stromal and epithelial cells and within the ECM at days 2 and 3. It was distinctly evident at the apical surface of epithelial cells at days 4 and 5 of pregnancy. Concurrent with this apical localization, bFGF was present in the uterine luminal fluid, suggesting release of this growth factor from epithelial cells. Embryonic implantation was accompanied by increased intracellular bFGF content in luminal epithelial and decidual cells. However, similar cells outside of the implantation site and in the artificially decidualized uterus did not express analogous bFGF levels, indicating that a unique signal from the embryo triggers bFGF expression. Changes in the cell-specific distribution of bFGF imply a multifunctional role for this growth factor in uterine cell proliferation, differentiation, and embryonic implantation. In addition, the apical release of bFGF from epithelial cells indicates utilization of a novel secretory pathway for bFGF export during early pregnancy [Carlone D L, Rider V; Biol. Rerod. 1993, 49(4): 653-65]. In the mouse, FGF signaling induces the cell division of embryonic and extra embryonic cells in the preimplantation embryo starting at the fifth cell division [Chai N et al., Dev Biol 1998, 198(1): 105-15]. bFGF is present within the implantation chamber on days 6-9 of pregnancy and may be involved in the decidual cell response, trophoblast cell invasion and angiogenesis [Wordinger R J et al., Growth factors. 1994, 11(3): 175-86].

It has been hypothesized for some time that secretions of the oviduct and uterus are involved in stimulating cell proliferation in preimplantation mammalian embryos and promotion of early differentiation events that lead to successful implantation. At least some of the regulatory factors present within uterine secretions are growth factors that can act along a paracrine pathway by binding to specific receptors on embryonic cells. Perhaps, then, in addition to functions of growth factors acting singly on their specific receptors, combinations of factors are important for induction of a specific developmental response. It is also possible that the result of combinations of factors may involve a process of interference whereby exposure of embryonic cells to one growth factor may compromise its ability to bind and respond to another [Schulz G A, Heyner S; Oxf. Rev. Reprod. Biol. 1993, 15: 43-81].

Expression of heparanase encoding DNA (hpa) in animal cells: As shown in U.S. patent application Ser. No. 09/071,618, filed May 1, 1998, which is incorporated herein by reference, transfected CHO cells expressed the hpa gene products in a constitutive and stable manner. Several CHO cellular clones have been particularly productive in expressing hpa proteins, as determined by protein blot analysis and by activity assays. Although the hpa DNA encodes for a large 543 amino acids protein (expected molecular weight of about 60 kDa) the results clearly demonstrate the existence of two proteins, one of about 60 kDa (p60) and another of about 45-50 kDa (p45). It has been previously shown that a 45-50 kDa protein with heparanase activity was isolated from placenta [Goshen, R. et al. Mol. Human Reprod. 1996, 2: 679-684] and from platelets [Freeman and Parish Biochem. J. 1998, 339:1341-1350]. It is thus likely that the 60 kDa protein is the pro-enzyme (i.e., proheparanase), which is naturally processed in the host cell to yield the 45-50 kDa protein (i.e., processed heparanase). The p45 was found to be at least 10 fold more active than the p60 protein, suggesting that p45 is the active enzyme. In addition, high five insect cells were transfected using recombinant baculovirus containing the hpa gene. These cells produced only the 60 kDa form of heparanase.

### SUMMARY OF THE INVENTION

While reducing the present invention to practice it was discovered that (i) heparanase adheres to the extracellular matrix of cells; (ii) cells to which heparanase is externally adhered process the heparanase to an active form; (iii) cells to which an active form of heparanase is externally adhered protect the adhered heparanase from the surrounding medium; (iv) cells to which an active form of heparanase is externally adhered, either cells genetically modified to express and secrete heparanase, or cells to which purified heparanase has been externally added are much more readily translocatable within the body as compared to cells devoid of externally adhered heparanase. It has been therefore realized that heparanase, as well as other extracellular matrix degrading enzymes, can be used to assist in introduction of biological materials, such as cells, tissues and drug delivery systems into patients.

While reducing the present invention to practice it was further discovered that heparanase induces bone formation and hence can be used to induce bone formation in cases where acceleration of bone formation is advantageous, such as in medical conditions characterized by bone loss, including osteogenesis imperfecta, osteoporosis, mucopolysaccharidoses associated bone defects, bone fractures, periodontal disease, other diseases characterized by bone loss or bone void formation, etc.

Thus, according to one aspect of the present invention there is provided biological preparation comprising a biological material and a purified, natural or recombinant, extracellular matrix degrading enzyme being externally adhered thereto. The biological material can be a plurality of cells, such as, marrow hematopoietic or stromal stem cells, keratinocytes, blastocysts, neuroblasts, astrocytes, fibroblasts and cells genetically modified with a therapeutic gene. Alternatively, the biological material is a tissue or a portion thereof, such as, embryo, skin flaps and bone scraps. Still alternatively, the biological material can be a drug delivery system.

According to another aspect of the present invention there are provided genetically modified cells expressing and secreting a recombinant extracellular matrix degrading enzyme, the extracellular matrix degrading enzyme being externally adhered thereto.

According to still another aspect of the present invention there are provided pharmaceutical composition comprising the above biological preparation or cells in combination with a pharmaceutically acceptable carrier.

According to yet another aspect of the present invention there is provided an in vivo method of repairing a tissue, such as, bone, muscle, skin or nerve tissue, the method comprising the steps of (a) providing cells capable of proliferating and differentiating in vivo to form the tissue or a portion thereof, the cells having an extracellular matrix degrading enzyme externally adhered thereto; and (b) administering the cells in vivo. The enzyme is either externally added to the cells, or alternatively, the cells are genetically modified to express and extracellularly present or secrete the enzyme.

According to still another aspect of the present invention there is provided an in vivo method of implanting a tissue, such as embryo, skin flaps or bone scraps, or a portion thereof, the method comprising the steps of (a) externally adhering to the tissue or the portion thereof a purified, natural or recombinant extracellular matrix degrading enzyme; and (b) implanting the tissue or the portion thereof in vivo.

According to an additional aspect of the present invention there is provided an in vivo method of cell transplantation, the method comprising the steps of (a) providing transplantable cells, such as bone marrow hematopoietic or stromal stem cells, keratinocytes, blastocysts, neuroblasts, astrocytes or fibroblasts, the cells having an extracellular matrix degrading enzyme externally adhered thereto; and (b) administering the cells in vivo. The enzyme is either externally added to the cells, or alternatively, the cells are genetically modified to express and extracellularly present or secrete the enzyme.

According to yet an additional aspect of the present invention there is provided a somatic gene therapy method of in vivo introduction of genetically modified cells expressing a therapeutic protein capable of relieving symptoms of a genetic disease such as mucopolysaccharidoses, cystic fibrosis, Parkinsohn's disease, Gaucher's syndrome or osteogenesis imperfecta, the method comprising the steps of (a) providing the genetically modified cells expressing the therapeutic protein, such as bone marrow hematopoietic or stromal stem cells, keratinocytes, blastocysts, neuroblasts, astrocytes or fibroblasts, having an extracellular matrix degrading enzyme externally adhered thereto; and (b) administering the cells in vivo. The enzyme is either externally added to the cells, or alternatively, the cells are genetically modified to express and extracellularly present or secrete the enzyme.

According to still an additional aspect of the present invention there is provided a method of delivering a biological material across a biological blood barrier, such as a blood-brain-barrier, a blood-milk-barrier or a maternal blood-placenta-embryo barrier, the method comprising the steps of (a) externally adhering to the biological material a purified, natural or recombinant, extracellular matrix degrading enzyme; and (b) administering the biological material in vivo. The biological material can be a plurality of cells or a drug delivery system.

According to a further aspect of the present invention there is provided a method of delivering cells across a biological blood barrier, such as a blood-brain-barrier, a blood-milk-barrier or a maternal blood-placenta-embryo barrier, the method comprising the steps of (a) genetically modifying the cells to express and extracellularly present or secrete an extracellular matrix degrading enzyme; and (b) administering the cells in vivo.

According to yet a further aspect of the present invention there is provided a method of managing a patient having an accumulation of mucoid, mucopurulent or purulent material containing glycosaminoglycans, the method comprising the step of administering at least one glycosaminoglycans degrading enzyme to the patient in an amount therapeutically effective to reduce at least one of the following: the visco-elasticity of the material, pathogens infectivity and inflammation, the at least one glycosaminoglycans degrading enzyme being administered in an inactive form and being processed by proteases inherent to the mucoid, mucopurulent or purulent material into an active form.

According to further features in preferred embodiments of the invention described below, the extracellular matrix degrading enzyme can be, for example, a collagenase (i.e., a metaloproteinase), a glycosaminoglycans degrading enzyme and an elastase. The glycosaminoglycans degrading enzyme can be, for example, a heparanase, a connective tissue activating peptide, a heparinase, a glucoronidase, a heparitinase, a hyluronidase, a sulfatase and a chondroitinase. The extracellular matrix degrading enzyme can be in an inactive form which is processed to be active by endogenous proteases. Alternatively, the extracellular matrix degrading enzyme can be in its active form.

According to another aspect of the present invention there is provided a method of inducing bone formation in a subject in need thereof, the method comprising administering to the subject a therapeutically effective amount of heparanase.

According to yet another aspect of the present invention there is provided a pharmaceutical composition for inducing bone formation in a subject in need thereof, the pharmaceutical composition comprising as an active ingredient a therapeutically effective amount of heparanase, and a pharmaceutically acceptable carrier.

According to further features in preferred embodiments of the invention described below, the heparanase is a proheparanase.

According to still further features in the described preferred embodiments the heparanase is a processed heparanase.

According to still further features in the described preferred embodiments the subject has a medical condition characterized by bone loss, reduced bone mass or bone fracture.

According to still further features in the described preferred embodiments the pharmaceutical is contained in a container and identified in print for use in a subject having a medical condition characterized by bone loss, reduced bone mass or bone fracture.

According to still further features in the described preferred embodiments the subject has osteogenesis imperfecta.

According to still further features in the described preferred embodiments the pharmaceutical composition is contained in a container and identified in print for use in a subject having osteogenesis imperfecta.

According to still further features in the described preferred embodiments the subject has osteoporosis.

According to still further features in the described preferred embodiments the pharmaceutical composition is contained in a container and identified in print for use in a subject having osteoporosis.

According to still further features in the described preferred embodiments the subject has mucopolysaccharidoses associated bone defect.

According to still further features in the described preferred embodiments the pharmaceutical composition is contained in a container and identified in print for use in a subject having mucopolysaccharidoses associated bone defect.

According to still further features in the described preferred embodiments the subject has a periodontal disease.

According to still further features in the described preferred embodiments the pharmaceutical composition is contained in a container and identified in print for use in a subject having periodontal disease According to still further features in the described preferred embodiments the subject has a bone void.

According to still further features in the described preferred embodiments the pharmaceutical composition is contained in a container and identified in print for use in a subject having a bone void.

According to still further features in the described preferred embodiments administering is via intrabone injection.

According to still further features in the described preferred embodiments the pharmaceutical composition is formulated for administration via intrabone injection.

The present invention successfully addresses the shortcomings of the presently known configurations by providing new tools for efficient introduction of cells, tissues and drug delivery systems into patients. The present invention successfully addresses the shortcomings of the presently known configurations by providing new tools for inducing bone formation.

### BRIEF DESCRIPTION OF THE DRAWINGS

The invention herein described, by way of example only, with reference to the accompanying drawings, wherein:

FIGS. 1a-b demonstrate that cells protect heparanase from inactivation by the surrounding pH and the presence of serum. The degradation of radiolabeled-ECM was tested, following the addition of heparanase to culture media, in the absence (1a), or presence (1b) of bone marrow stem cells (BMSC). 1a—Either heparanase or buffer (0.4 M NaCl, 20 mM buffer phosphate pH-6.8) were added to radiolabeled ECM plates in DMEM+10% FCS, the pH of the media was measured, and the activity of heparanase was tested. ss71=substrate, Cs buffer, Es heparanase. 1b—BMSCs were grown on radiolabeled ECM plates and the presence of degraded radiolabeled ECM products in the growth media was tested before and after the addition of buffer (1), or heparanase (2).

FIGS. 2a-b demonstrate that heparanase adheres to BMSCs and retains its activity. Cells that were incubated with heparanase were washed, collected and subjected to the (2a) DMB heparanase activity assay (1-6 represent six different experiments) and (2b) Western blot analysis using anti heparanase antibodies. T=Trypsin, 1E=1 mM EDTA, 2E=2 mM EDTA, Cb=control, purified heparanase from baculovirus, p60, Cc=control, purified heparanase from CHO cells, p45, kDa=kiloDaltons.

FIG. 3 demonstrates that the presence of GAGs is required for heparanase adherence to cells. Cells were incubated with heparanase for 2 hours, washed, collected and subjected to the DMB heparanase activity assay.

FIGS. 4a-c demonstrate that heparanase adheres to B16-F1 cells and retain its activity. Cells that were either transfected with the hpa cDNA (“transfected”), or incubated with heparanase (“adhered”, +b22, or +b27), or not treated with heparanase (NT or −), were washed, collected and subjected to the DMB heparanase activity assay (4a), gel shift assay (4b), and Western blot analysis using anti heparanase antibodies (4c). Purified baculovirus heparanase p60 (b22, b27), or CHO heparanase p45 were used as controls (C).

FIGS. 5a-b demonstrate that heparanase binds to CHO-dhfr cell line, undergoes proteolytic cleavage and exhibits high heparanase activity. Cells that were incubated with heparanase were washed, collected and subjected to DMB activity assay (5a), and Western blot analysis using anti-heparanase antibodies (5b).

FIGS. 6a-c demonstrate the effect of sputum-proteases on the proteolytic activation of heparanase. (6a) The effect of heparanase on sputum viscosity was tested using microviscosometer. (6b) The reduction of the volume of sputum solids, in sputum samples that were incubated 2 hours at 37° C., with either baculovirus derived heparanase—p60 (Nos. 1 and 2), or saline (Nos. 3 and 4), or CHO p45 heparanase (Nos. 5 and 6), as well as with (No. 8) or without (No. 7) p60 heparanase, in the presence of protease inhibitors (PI), was observed following centrifugation, and the supernatants were subjected to Western blot analysis (6c) using 2 different anti-heparanase monoclonal antibodies: No. 239 which recognizes only the p60 form, and No. 117 which recognizes both the p60 and the p45 forms.

FIG. 7 demonstrate the effect of heparanase on tumor cell metastasis, in vivo. C57BL mice were injected by B16-F1 melanoma cells that, were either transfected by the Hpa cDNA (“transfect”), or coated with the p60-heparanase enzyme (“adhered”), either without or with fragmin (“I”). The number of metastases in the lungs was counted 3 weeks post-injection.

FIGS. 8a-g demonstrate the effect of heparanase on the formation of bone like-tissue from primary BMSC cultures. FIGS. 8a-b- the effect of heparanase on BMSCs proliferation was measured for two independent rats using the MTT proliferation test. The control, cells at day zero, was calculated as 100%. FIGS. 8c-d- the effect of heparanase on BMSCs state of differentiation was determined for the above mentioned rats, respectively, by alkaline phosphatase (ALP) activity. The relative ALP activity as compared to the total protein was also calculated (8e). FIGS. 8f-g—the effect of heparanase on BMSCs mineralization was determined for the above rats, respectively, and expressed by the relative stained area of the well.

### DESCRIPTION OF THE PREFERRED EMBODIMENTS

The present invention is of methods, preparations and pharmaceutical compositions which can be used to assist in introduction of biological materials, such as cells, tissues and drug delivery systems into patients. Specifically, the present invention can be used to improve processes involving implantation and transplantation of a variety of cells and tissues in cases of, for example, somatic gene therapy or cells/tissues implantations/transplantation. The present invention is further of a method and pharmaceutical composition for inducing bone formation.

The principles and operation of the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

As exemplified in the Examples section that follows, while reducing the present invention to practice it was discovered that when externally added, heparanase adheres to cells. It was further discovered that cells to which heparanase is externally adhered to process the heparanase to an active form and that cells to which an active form of heparanase is externally adhered protect the adhered heparanase from the surrounding medium, such that the adhered heparanase retains its catalytic activity under conditions which otherwise hamper its activity. It was further discovered that cells to which an active form of heparanase is externally adhered, either cells genetically modified to express and extracellularly present or secrete heparanase, or cells to which purified heparanase has been externally added, are much more readily translocatable within the body of experimental animal models, as compared to cells devoid of externally adhered heparanase. Additional discoveries made while reducing the present invention to practice show that inactive pro-heparanase can be processed by endogenous proteases into its active form. Still additional discoveries made while reducing the present invention to practice show that heparanase can be used to induce bone formation.

It has been therefore realized that heparanase, as well as other extracellular matrix degrading enzymes, can be used to assist in introduction of biological materials, such as cells, tissues and drug delivery systems into desired locations in the bodies of patients. It has been further realized that heparanase, as well as other extracellular matrix degrading enzymes, can be used to assist in bone formation in medical conditions characterized by bone loss, including osteogenesis imperfecta, osteoporosis, mucopolysaccharidoses associated bone defects, bone fractures, periodontal disease, other diseases characterized by bone loss or bone void formation, etc.

As used herein in the specification and in the claims section below, the term “heparanase” refers to an animal endoglycosidase hydrolyzing enzyme which is specific for heparin or heparan sulfate proteoglycan substrates, as opposed to the activity of bacterial enzymes (heparinase I, II and III) which degrade heparin or heparan sulfate by means of β-elimination. The heparanase can be natural or recombinant and optionally modified, precursor or activated form, as described in U.S. Pat. No. 6,348,344, entitled GENETICALLY MODIFIED CELLS AND METHODS FOR EXPRESSING RECOMBINANT HEPARANASE AND METHODS OF PURIFYING SAME, which is incorporated herein by reference.

As used herein in the specification and in the claims section below, the phrase “drug delivery system” include liposomes, granules and the like which include an inner volume containing a drug which is thereafter released therefrom. Such liposomes and granules are well known in the art. Such liposomes, for example, can be manufactured having glycolipids and/or glycoproteins embedded therein, so as to create an artificial extracellular matrix to which extracellular matrix degrading enzymes can adhere.

According to one aspect of the present invention there is provided a biological preparation which includes a biological material and a purified, natural or recombinant, extracellular matrix degrading enzyme which is externally adhered to the biological material. The biological material according to this aspect of the present invention can be a plurality of cells, such as, but not limited to, marrow hematopoietic or stromal stem cells, keratinocytes, blastocysts, neuroblasts, astrocytes, fibroblasts and cells genetically modified with a therapeutic gene producing a therapeutic protein. Alternatively, the biological material is a tissue or a portion thereof, such as, but not limited to, an embryo, skin flaps or bone scraps. Still alternatively, the biological material can be a drug delivery system.

As used herein in the specification and in the claims section below, the term “externally adhered” refers to associated with, e.g., presented. When applies to cells (or tissues) it refers to associated with the extracellular matrix. It will be appreciated that some cells/tissues have inherent extracellular matrix degrading enzyme(s) adhered thereto. The present invention, however, is directed at adding additional adhered enzyme thereto by man intervention.

As used herein in the specification and in the claims section below, the term “purified” includes also enriched. Methods of purification/enrichment of extracellular matrix degrading enzyme are well known in the art. Examples are provided in U.S. patent application Ser. No. 09/071,618, filed May 1, 1998, in Goshen et al. [Goshe R et al. Mol. Human Reprod. 2, 679-684, 1996] and in WO91/02977, which are incorporated herein by reference.

As used herein in the specification and in the claims section below, the term “natural” refers to an enzyme of a natural origin.

As used herein in the specification and in the claims section below, the term “recombinant” refers to an enzyme encoded by a gene introduced into an expression system.

As used herein in the specification and in the claims section below, the term “enzyme” refers both to the inactive pro-enzyme form and to its processed active form.

According to another aspect of the present invention there are provided genetically modified cells expressing and extracellularly presenting or secreting a recombinant extracellular matrix degrading enzyme, the extracellular matrix degrading enzyme is externally presented or adhered to the cells.

As used herein in the specification and in the claims section below, the phrase “genetically modified” refers to cells which incorporate a recombinant nucleic acid sequence.

According to still another aspect of the present invention there are provided pharmaceutical composition which contain the above biological preparation or cells in combination with a pharmaceutically acceptable carrier, such as thickeners, buffers, diluents, surface active agents, preservatives, and the like, all as well known in the art. A pharmaceutical composition according to the present invention may also include one or more active ingredients, such as but not limited to, anti inflammatory agents, anti microbial agents, anesthetics and the like.

The pharmaceutical composition according to the present invention may be administered in either one or more of ways depending on whether local or systemic treatment is of choice, and on the area to be treated. Administration may be done topically (including ophtalmically, vaginally, rectally, intranasally), orally, by inhalation, or parenterally, for example by intravenous drip or intraperitoneal, subcutaneous, intramuscular or tissue specific injection, such as, but not limited to, intrauterine, intratrachea, intramammary gland, intrabrain or intrabone injection.

Formulations for topical administration may include, but are not limited to, lotions, ointments, gels, creams, suppositories, drops, liquids, sprays and powders. Conventional pharmaceutical carriers, aqueous, powder or oily bases, thickeners and the like may be necessary or desirable. Compositions for oral administration include powders or granules, suspensions or solutions in water or non-aqueous media, sachets, capsules or tablets. Thickeners, diluents, flavorings, dispersing aids, emulsifiers or binders may be desirable. Formulations for parenteral administration may include, but are not limited to, sterile aqueous solutions which may also contain buffers, diluents and other suitable additives.

Dosing is dependent on severity and responsiveness of the condition to be treated, but will normally be one or more doses with course of treatment lasting from several days to several months or until a cure is effected or a diminution of disease state is achieved. Persons ordinarily skilled in the art can easily determine optimum dosages, dosing methodologies and repetition rates.

The preparations, cells and pharmaceutical compositions according to the present invention can be used to implement several therapeutic protocols as, for example, further detailed in the following sections.

Thus, according to yet another aspect of the present invention there is provided an in vivo method of repairing a tissue or a portion thereof, such as, but not limited to, a damaged bone, muscle, skin or nerve tissue. The method according to this aspect of the invention is effected by providing cells capable of proliferating and differentiating in vivo to form and therefore repair the tissue or a portion thereof, the cells have an extracellular matrix degrading enzyme externally adhered thereto, and administering the cells in vivo. The enzyme is either externally added to the cells, or alternatively, the cells are genetically modified to express and extracellularly present or secrete the enzyme. As is exemplified in the Examples section that follows, such cells are much more readily arriving and established in the receptive tissue.

According to still another aspect of the present invention there is provided an in vivo method of implanting a tissue, such as, but not limited to, embryo, skin flaps or bone scraps. The method according to this aspect of the present invention is effected by externally adhering to the tissue or to a portion thereof a purified, natural or recombinant, extracellular matrix degrading enzyme, and implanting the tissue or the portion thereof in vivo.

According to an additional aspect of the present invention there is provided an in vivo method of cell transplantation. The method according to this aspect of the present invention is effected by providing transplantable cells, such as bone marrow hematopoietic or stromal stem cells, keratinocytes, blastocysts, neuroblasts, astrocytes, fibroblasts, the cells have an extracellular matrix degrading enzyme externally adhered thereto, and administering the cells in vivo. The enzyme according to this aspect of the invention is either externally added to the cells, or alternatively, the cells are genetically modified to express and extracellularly present or secrete the enzyme. This method can be used, for example, to transplant cells of a healthy donor in an MHC matching patient which suffers from a genetic disease, characterized, for example, in a deficiency of a protein.

According to yet an additional aspect of the present invention there is provided a somatic gene therapy method of in vivo introduction of genetically modified cells expressing a therapeutic protein capable of relieving symptoms of a genetic disease, such as, but not limited to, mucopolysaccharidoses, cystic fibrosis, Parkinsohn's disease, Gaucher's syndrome or osteogenesis imperfecta. The method according to this aspect of the present invention is effected by providing the genetically modified cells expressing the therapeutic protein (e.g., bone marrow hematopoietic or stromal stem cells, keratinocytes, blastocysts, neuroblasts, astrocytes and fibroblasts) and having an extracellular matrix degrading enzyme externally adhered thereto, and administering the cells in vivo. As before, the enzyme is either externally added to the cells, or alternatively, the cells are genetically modified to express and extracellularly present or secrete the enzyme.

According to still an additional aspect of the present invention there is provided a method of delivering a biological material across a biological blood barrier, such as, but not limited to, a blood-brain-barrier, a blood-milk-barrier or a maternal blood-placenta-embryo barrier. The method according to this aspect of the present invention is effected by externally adhering to the biological material a purified, natural or recombinant, extracellular matrix degrading enzyme, and administering the biological material in vivo. The biological material can be a plurality of cells or a drug delivery system.

According to a further aspect of the present invention there is provided a method of delivering cells across a biological blood barrier. The method according to this aspect of the present invention is effected by genetically modifying the cells to express and extracellularly present or secrete an extracellular matrix degrading enzyme and administering the cells in vivo.

According to yet a further aspect of the present invention there is provided a method of managing a patient having an accumulation of mucoid, mucopurulent or purulent material containing glycosaminoglycans. The method according to this aspect of the present invention is effected by administering at least one glycosaminoglycans degrading enzyme to the patient in an amount therapeutically effective to reduce at least one of the following: the visco-elasticity of the material, pathogens infectivity and inflammation, the at least one glycosaminoglycans degrading enzyme being administered in an inactive form and being processed by proteases inherent to the mucoid, mucopurulent or purulent material into an active form.

The extracellular matrix degrading enzyme which can be used to implement the above described therapeutic methods according to the present invention can be, for example, a collagenase (i.e., a metaloproteinase), a glycosaminoglycans degrading enzyme and an elastase. The glycosaminoglycans degrading enzyme can be, for example, a heparanase, a connective tissue activating peptide, a heparinase, a glucoronidase, a heparitinase, a hyluronidase, a sulfatase and a chondroitinase. The extracellular matrix degrading enzyme can be in an inactive form which is processed to be active by endogenous proteases. Alternatively, the extracellular matrix degrading enzyme can be in its active form. These enzymes and others are available in an enriched form from various sources. The genes encoding these enzymes have been cloned, such that recombinant enzymes are either available or can be readily made available.

According to another aspect of the present invention there is provided a method of inducing bone formation in a subject in need thereof. The method according to this aspect of the present invention finds experimental support and is further described in the Examples section that follows under the title “The effects of heparanase on bone formation” and comprises administering to the subject a therapeutically effective amount of heparanase.

According to yet another aspect of the present invention there is provided a pharmaceutical composition for inducing bone formation in a subject in need thereof. The pharmaceutical composition according to this aspect of the invention comprises, as an active ingredient, a therapeutically effective amount of heparanase, and a pharmaceutically acceptable carrier.

The heparanase used in the method and pharmaceutical composition can be a proheparanase, which upon administration, will be processed by natural proteases into processed heparanase, or the heparanase used in the method and pharmaceutical composition can be processed heparanase, in which case no further in vivo processing is required.

The method and pharmaceutical composition described herein can be used to treat any medical condition characterized by bone loss, reduced bone mass or bone fracture. Examples include osteogenesis imperfecta, osteoporosis, mucopolysaccharidoses associated bone defects and a periodontal disease characterized by bone loss.

The pharmaceutical composition is preferably contained in a container and identified in print for use in a subject having any one of the above medical conditions/diseases.

A presently preferred mode of administration of heparanase or a pharmaceutical composition containing same to a treated bone is via intrabone injection. To this end, the pharmaceutical composition is preferably formulated for administration via intrabone injection.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures in recombinant DNA technology described below are those well known and commonly employed in the art. Standard techniques are used for cloning, DNA and RNA isolation, amplification and purification. Generally enzymatic reactions involving DNA ligase, DNA polymerase, restriction endonucleases and the like are performed according to the manufacturers' specifications. These techniques and various other techniques are generally performed according to Sambrook et al., molecular Cloning—A Laboratory Manual, Cold Spring Harbor Laboratory, Cold Spring Harbor, N.Y. (1989). The manual is hereinafter referred to as “Sambrook”. Other general references are provided throughout this document. The procedures therein are believed to be well known in the art and are provided for the convenience of the reader. All the information contained therein is incorporated herein by reference.

### Materials And Experimental Methods

Cells:

Bone Marrow Stromal Cells (BMSCs): Femurs form 2 male, 45 days old, Sprague-Dawley rats, were obtained from Harlan Biotech Israel, in a sterile manner, and shipped in saline at 30° C. (rat No. 1) and at 4° C. (rat No. 2). Bone marrow cells were flushed out, pooled (from 2 femurs of one rat), and cultured in MEMα, containing 15% heat inactivated FCS, Penicillin/Streptomycin—100u/100 μg per ml, 2 mM Glutamine, 0.25 mg/ml Fungizone (all purchased from Beit Haemek, Israel), 10 mM β-glycerolphosphate, ascorbic acid 50 μg/ml (Sigma) and 10⁻⁷ M dexamethasone (Vitamed). Cultures were maintained in a humidified, 8% CO₂, 37° C., incubator. Following 3 days of incubation, non-adhered cells were washed out, and the adherent cells were re-cultured in the complete MEMα medium. The medium was changed every two days for a week thereafter. Then, the cells were trypsinized and counted. Cells were subcultured into 11 96 well plates. One plate was subjected to MTT proliferation test (see hereinunder), and the rest of the plates were maintained in a humidified, 8% CO₂, 37° C., incubator in complete MEMα medium with 10⁻⁸ M dexamethasone. On days 12 and 15, a plate was subjected for each and every of the following tests: MTT, alkaline phosphatase and alizarin red staining. An MTT test was also done on day 6.

CHO cells: CHO cells and CHO sublines No. 803, which expresses only very little heparan sulfate, and No. 745 which expresses only very little glycosaminoglycans [Esko J D et al., Science 1988, 241: 1092-6], were cultured in either DMEM or F12 containing 10% heat inactivated FCS (Beit-Haemek).

B16-F1 cells: B16-F1 cells were cultured in DMEM +10% FCS.

MTT-Cell Proliferation Test:

Cells were washed three times with RPMI (Beit Haemek). MTT (Thiazolyl blue, Cat. No. M5655, Sigma) was dissolved in RPMI at concentration of 1 mg/ml and filtered through an 0.2 μm filter. 100 μl of the filtrate were added to each well. Following 3 hours of incubation at 37° C., 100 μl of stop solution (50% DMF, 10% SDS, 2% acetic acid, and 0.025N HCl, all from Sigma) was added to each well, and plate(s) were incubated overnight at room temperature. Color formation was determined using ELISA reader at 580 nm.

Alkaline phosphatase activity (ALP): Cells were washed three times with Dulbeco's PBS× 1 (Beit Haemek), followed by addition of 0.5 ml of 10 mM Tris-HCl buffer, pH-7.6, containing 10 mM MgCl₂ and 0.1% Triton. Cells were then freezed and thawed three times and stored at −20° C. An alkaline phospatase activity kit was purchased from Sigma. When ready to analyze, 5 μl of cell lysates from each well were incubated with 200 ml of the supplied substrate. The absorbency was determined at 405 nm by ELISA reader, every one minute. ALP activity was calculated as described by the kit's distributor (Sigma).

Total protein determination (TP): From the above lysates, 5 μl were added to 200 μl Bradford reagent (BioRad), and the absorbency was determined at 580 nm by ELISA reader.

Alizarin red S staining: Cells were washed three times with Dulbeco's PBS× 1 (Beit Haemek), and then fixed overnight in methanol:formaldehyde:H₂O, at a ratio of 1:1:1.5. The wells were then washed and stained for 5 minutes with saturated solution of Alizrin red S (Sigma) pH-4.0. The wells were then washed and air dried.

Heparanase adherence to cells: Enzyme preparations used were purified recombinant heparanase of approximately 60 kDa expressed in insect cells (see U.S. patent application Ser. No. 09/071,618, filed May 1, 1998). The adherence of heparanase to cells was performed as follows: cells were plated in either 35 or 90 mm plates with antibiotic free DMEM or F12 media supplemented with 10% FCS. Following at least 24 hours of incubation in antibiotic-free media, 10 μg/ml of recombinant heparanase from baculovirus were added to cell culture, and incubated for 2 hours at 37° C. The plates were then washed twice with PBS, harvested by very short trypsinization, washed with PBS, and the pellet was either subjected to activity assay or Western blot analysis, or resuspended and injected into mice.

Western blot analysis: Proteins were separated on 4-20%, polyacrylamid ready gradient gels (Novex). Following electrophoresis proteins were electrotransferred to Hybond-P nylon membrane (Amersham, 350 mA/100V for 90 minutes). Membranes were blocked in TBS containing 0.02% Tween 20 and 5% skim milk for 1-16 hours, and then incubated with antisera or purified antibodies diluted in blocking solution. Blots were then washed in TBS-Tween, incubated with appropriate HRP-conjugated anti mouse/anti rabbit IgG, and developed using ECL reagents (Amersham) according to the manufacturer's instructions.

Heparanase activity assay: Enzyme preparations were incubated with 100 μl of 50% heparin sepharose beads suspension (Pharmacia) in 0.5 ml eppendorf tubes on a head-over-tail shaker (37° C., 17 hours) in reaction mixtures containing 20 mM phosphate citrate buffer pH 5.4, 1 mM CaCl₂, and 1 mM NaCl, in a final volume of 200 μl. Enzyme preparations used were purified recombinant heparanase expressed in insect cells (see U.S. patent application Ser. No. 09/071,618, filed May 1, 1998). At the end of the incubation time, the samples were centrifuged for 2 minutes at 1000 rpm, and the products released to the supernatant due to the heparanase activity were analyzed using the Dimethylmethylene Blue calorimetric assay described in U.S. patent Ser. No. 09/113,168, filed Jul. 10, 1998, which is incorporated by reference as if fully set forth herein.

Dimethylmethylene Blue assay (DMB): Supernatants (100 μl) were transferred to plastic cuvettes. The samples were diluted to 0.5 ml with PBS plus 1% BSA. 1,9-Dimethylmethylene (Aldrich) was prepared (32 mg dissolved in 5 ml ethanol and diluted to 1 liter with formate buffer) and 0.5 ml was added to each sample. Absorbency of the samples was determined using a spectrophotometer (Cary 100, Varian) at 530 nm. To each sample, a control, in which the enzyme was added at the end of the incubation period, was included.

Gel shift assay: Baculovirus derived-heparanase or cell lysates, were incubated with 5 μg heparin in 20 mM citrate phosphate buffer pH 5.4 for 17 hours at 37° C. The samples were then loaded onto 4-20% polyacrylamid, ready to use gradient gel (Novex). The gel was stained with 50% methylene blue in ethanol for 10 minutes, and de-stained with water.

Heparanase Activity Assay on Radiolabeled ECM-Coated Plates:

Preparation of dishes coated with ECM: Bovine corneal endothelial cells (BCECs, second to fifth passage) were plated into 4-well plates at an initial density of 2×10⁵ cells/ml, and cultured in sulfate-free Fisher medium supplemented with 5% dextran T-40 for 12 days. Na₂³⁵SO₄ (μCi/ml) was added on day 1 and 5 after seeding and the cultures were incubated with the label without medium change. The subendothelial ECM was exposed by dissolving (5 minutes, room temperature) the cell layer with PBS containing 0.5% Triton X-100 and 20 mM NH₄OH, followed by four washes with PBS. The ECM remained intact, free of cellular debris and firmly attached to the entire area of the tissue culture dish.

Heparanase activity: Cells (1×10⁶/35-mm dish), cell lysates or conditioned media were incubated on top of ³⁵S-labeled ECM (18 hours, 37° C.) in the presence of 20 mM phosphate buffer (pH 6.2). Cell lysates and conditioned media were also incubated with sulfate labeled peak I material (10-20 μl). The incubation medium was collected, centrifuged (18,000× g, 4° C., 3 minutes), and sulfate labeled material was analyzed by gel filtration on a Sepharose CL-6B column (0.9×30 cm). Fractions (0.2 ml) were eluted with PBS at a flow rate of 5 ml/hour and counted for radioactivity using Bio-fluor scintillation fluid. The excluded volume (V_{O}) was marked by blue dextran and the total included volume (Vₜ) by phenol red. The latter was shown to co-migrate with free sulfate. Degradation fragments of heparan sulfate side chains were eluted from Sepharose 6B at 0.5<Kav<0.8 (peak II). A nearly intact HSPG released from ECM by trypsin—and, to a lower extent, during incubation with PBS alone—was eluted next to V_{O} (Kav<0.2, peak I). Recoveries of labeled material applied on the columns ranged from 85 to 95% in different experiments. Each experiment was performed at least three times and the variation of elution positions (Kav values) did not exceed ±15%.

Lung metastasis induction in vivo: This experiment included 5 test groups of 6 (1 group with 7) mice, and one control group (not injected) of 2 mice. The mice groups were injected with cells as described bellow: Group 1 mice were injected with B16-F1 cells (melanoma cell line); Group 2 mice were injected with human heparanase transfected B16-F1 cells; Group 3 mice were injected with human heparanase transfected B16-F1 cells to which fragmin was added; Group 4 mice were injected with B16-F1 cells to which heparanase was adhered; Group 5 mice were injected with B16-F1cells to which both heparanase and fragmin were added; Group 6 included non-injected control mice.

The injected cells were prepared as follows:

Group 1: B16-F1 cells were grown in DMEM+10% FCS (Beit Haemek). Cells were trypsinized, harvested and centrifuged. The pellet was washed with PBS and resuspended in PBS at 2.5×10⁵ cells/ml, total of 10⁶ in 4 ml for 10 mice. Aliquots were prepared: 2×1.5 ml and 1×1 ml in 2 ml screw cupped tubes.

Group 2: B16-F1 cells were transfected (Fugene, Boehringer-Mannheim) with the heparanase cDNA (see U.S. patent Ser. No. 09/071,739, filed May 1, 1998, which is incorporated by reference as if fully set forth herein). The cells were then collected and divided as described for Group 1 mice.

Group 3: Transfected B16-F1 were prepared as in Group 2. The cells were then collected, fragmin (Pharmacia) was added at a concentration of 1 mg/ml, and the cells were divided to aliquots as described for Group 1.

Group 4: Heparanase was adhered to B16-F1 cells: 3×10⁶ cells were plated in 8 ml of antibiotic free DMEM supplemented with 10% FCS. Following 24 hours of incubation, 80 μg of recombinant heparanase from baculovirus (final concentration of 10 μg/ml) were added to the cell culture, and incubated for 2 hours at 37° C. The plates were then washed twice with PBS, harvested by very short trypsinization, washed with PBS, and resuspended in PBS at 2.5×10⁵ cells/ml (total of 10⁶ in 4 ml for 10 mice). Aliquots were prepared: 2×1.5 ml, 1×1 ml in 2 ml screw cap tubes.

Group 5: Heparanase was adhered to cells as described for Group 4. The cells were then collected, fragmin was added at a concentration of 1 mg/ml, and cells were divided to aliquots as described for Group 1.

Quantitative Assessment of Lung Metastases:

Thirty three (33) adult C57BL male mice, weighing in the range of 17.1-26.9 at the time of study initiation, were supplied by Harlan Laboratories, Israel. Following receipt, animals were acclimated for eight days, during which they were observed daily for their condition and for signs of ill-health. Animals were kept within a limited access rodent facility, with environmental conditions set to a target temperature of 20±2° C., a target humidity of 30-70% and a 12 hours light/12 hours dark cycle. Temperature and relative humidity were monitored daily by the control computer. No deviations from the target values were observed.

Animals were housed during acclimation and test period in polypropylene cages, six animals per cage. Each cage was equipped with a cage card, visible on the front of the cage and containing all relevant details such as study number, sex, strain, etc.

Animals were provided ad libitum access to a commercial laboratory rodent diet (Harlan Teklad TRM Rat/Mouse Diet) and to drinking water, supplied to each cage via polyethylene bottles with stainless steel sipper-tubes.

Animals were arbitrarily assigned to the following test article and control groups as follows:

| Test | Group | |
|---|---|---|
| Group No. | size | Animal No. |
| 1 | n = 6 | 1, 2, 3, 4, 5, 6 |
| 2 | n = 6 | 7, 8, 9, 11, 12, 31 |
| 3 | n = 6 | 13, 14, 15, 16, 17, 18 |
| 4 | n = 6 | 19, 20, 21, 22, 23, 24 |
| 5 | n = 7 | 25, 26, 27, 28, 29, 30, 32 |
| 6* | n = 2 | 33, 34 |

*Control

Treated animals were subjected to a single intravenous administration of 0.4 ml/mouse of the above cell preparations injected via the tail vein.

Animals were observed for signs of ill health or reaction to treatment on the day of dosing and thereafter twice daily until study termination.

Body weight determinations were carried out just prior to dosing and thereafter on days 9, 13, 18 and at the time of study termination (day-2 1).

Determination of the number of lung metastases was performed in all animals, following euthanasia and excision of the lungs. Lung tissue was than rinsed in PBS, the individual lobes separated and subsequently the number of metastases counted under a binocular microscope. In the event metastases were observed in additional organs, they were likewise counted and recorded.

Sputum viscosity and proteolytic activation of heparanase by sputum-borne proteases: 250 μl of sputum samples, kept at 37° C., were mixed in eppendorf tubes with either recombinant heparanase (p60), or with saline, or with a cocktail of protease inhibitors followed by the addition of heparanase, to make a total volume of 350 μl. The samples were immediately transferred to 0.5 insulin syringes and tested for viscosity using a microviscosometer (Haake). The samples in the syringes were then incubated at 37° C. and tested again for viscosity after 10, 50 and 120 minutes. Then, the samples were centrifuged for 10 minutes at 13,000 rpm and the supernatants were subjected to Western blot analysis, using several anti-heparanase antibodies (monoclonal Nos. 117 and 239, described in U.S. patent application Ser. No. 09/071,739, filed May 1, 1998).

### Experimental Results

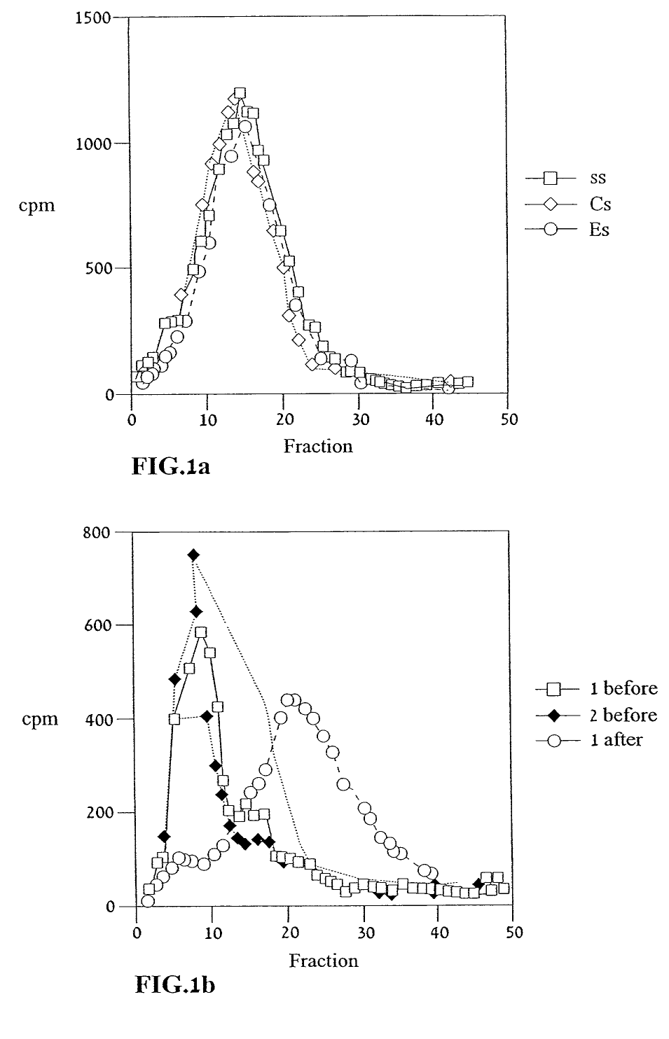

The adherence of heparanase to primary BMSC and various cell lines: In order to test the bioavailabilty and activity of heparanase in tissue culture conditions, as a prerequisite for in vivo clinical trials, recombinant human heparanase was added to radiolabeled-ECM plates in DMEM containing 10% FCS at pH>7.5. Under these conditions heparanase was not active as indicated by the absence of radiolabeled peak II which represents the heparanase degradation products (FIG. 1a). In contrast, when heparanase was added to radiolabeled ECM plates in DMEM containing 10% FCS at pH>7.5 in the presence of cultured bone marrow stromal cells (BMSC), heparanase was active as indicated by the presence of radiolabeled peak II (FIGS. 1b). It was, therefore, hypothesized that the cells protect the enzyme from the surrounding, thus enabling its activity.

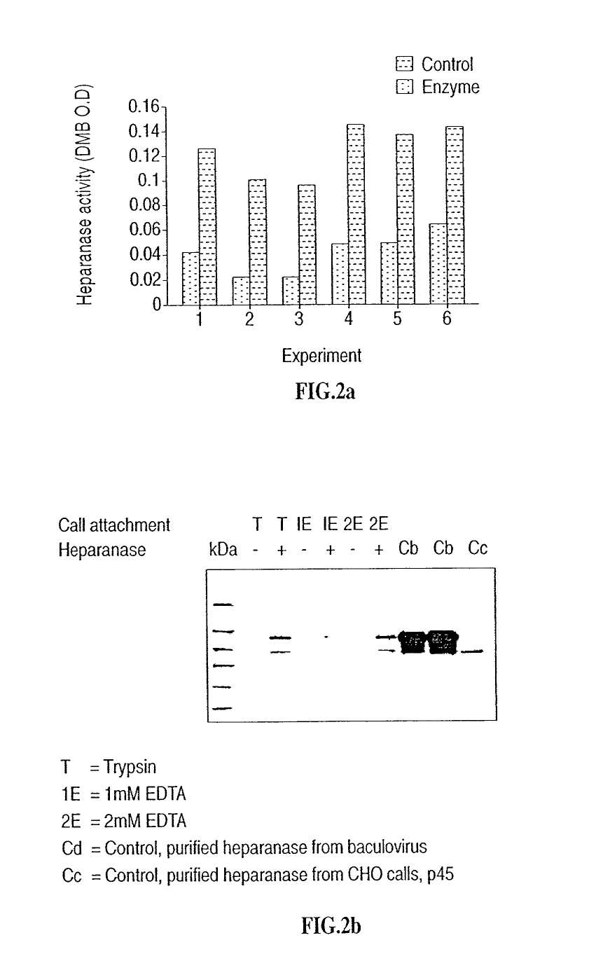

In order to test this hypothesis, heparanase (from baculovirus, p60, the pro-enzyme) was incubated with primary BMSC cultures. Following 2 hours of incubation, the cells were washed and heparanase activity was tested by the DMB assay. It was found that the cells exhibited a very high heparanase activity, whereas BMSCs do not posses heparanase activity, suggesting that the enzyme adhered to the cells and retained its activity (FIG. 2a).

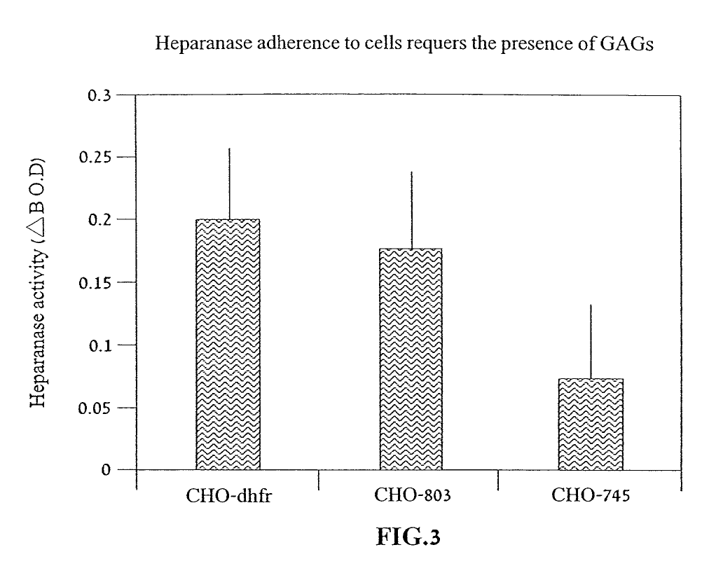

Next, it was interesting to find what is the ligand for heparanase? The following mutated CHO cell clones were incubated with heparanase: CHO cells (CHO-dhfr), CHO cells which express only very little heparan sulfate (HS, CHO-803), and CHO cells which express almost no GAGs (CHO-745, Esko JD et al., Science 1988, 241: 1092-6). It was found that the adherence of heparanase to the GAG-less cells was significantly decreased (FIG. 3).

These observations suggested that heparanase adheres to the cells via HS or other GAGs.

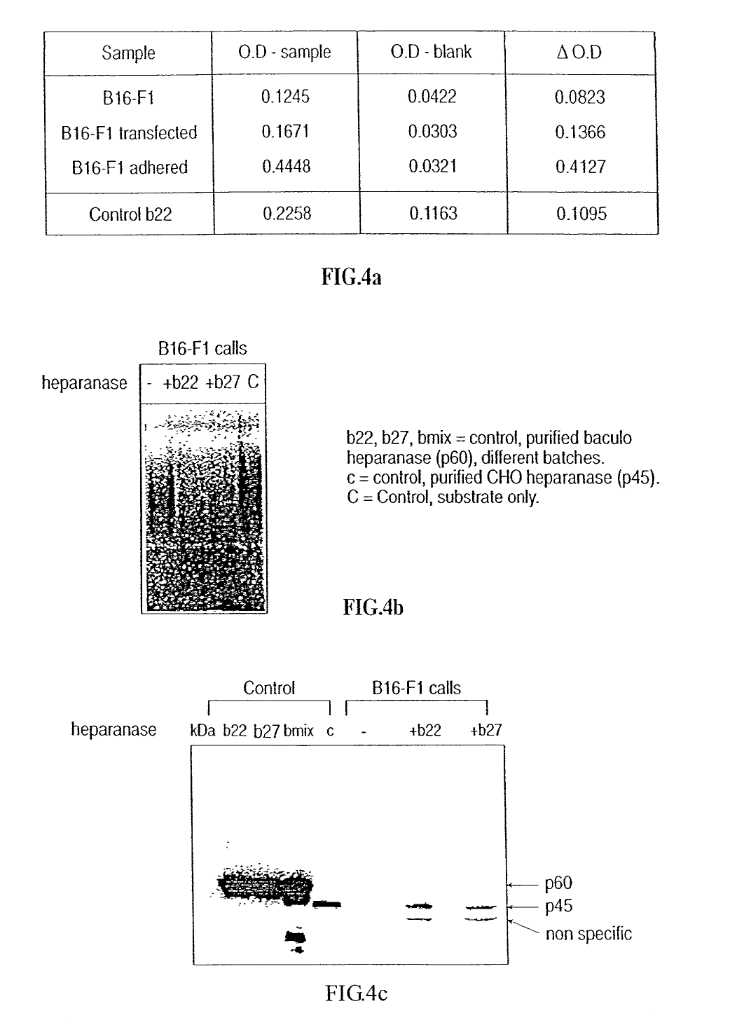

Furthermore, heparanase bound very efficiently to murine melanoma cells (B16-F1), and exhibited high heparanase activity (FIG. 4).

These results indicate that heparanase does not bind to a specific receptor, but rather binds to a more common type molecule(s).

In subconfluent cell monolayer the number of cells is proportional to cell size. For example, the approximate number of cells per 1 cm² of CHO subconfluent cell monolayer is 10⁵, for mouse lymphocytes subconfluent cell monolayer it is 4×10⁵, whereas for rat bone marrow stromal subconfluent cell monolayer it is 10⁴. This number of cells to which heparanase was adhered gives O.D.₅₃₀>0.1 in the heparanase DMB activity assay (U.S. patent application Ser. No. 09/113,168). However, using an equivalent number of cells, no measurable heparanase activity was detected in the DMB activity assay in rat bone marrow stromal cells and in mouse lymphocytes to which heparanase was not adhered.

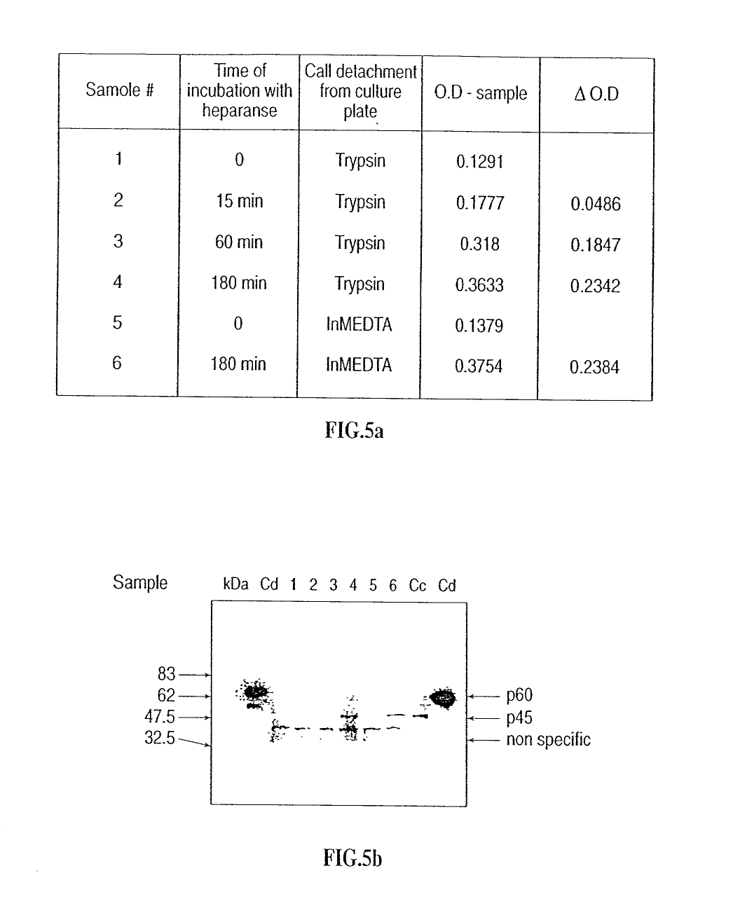

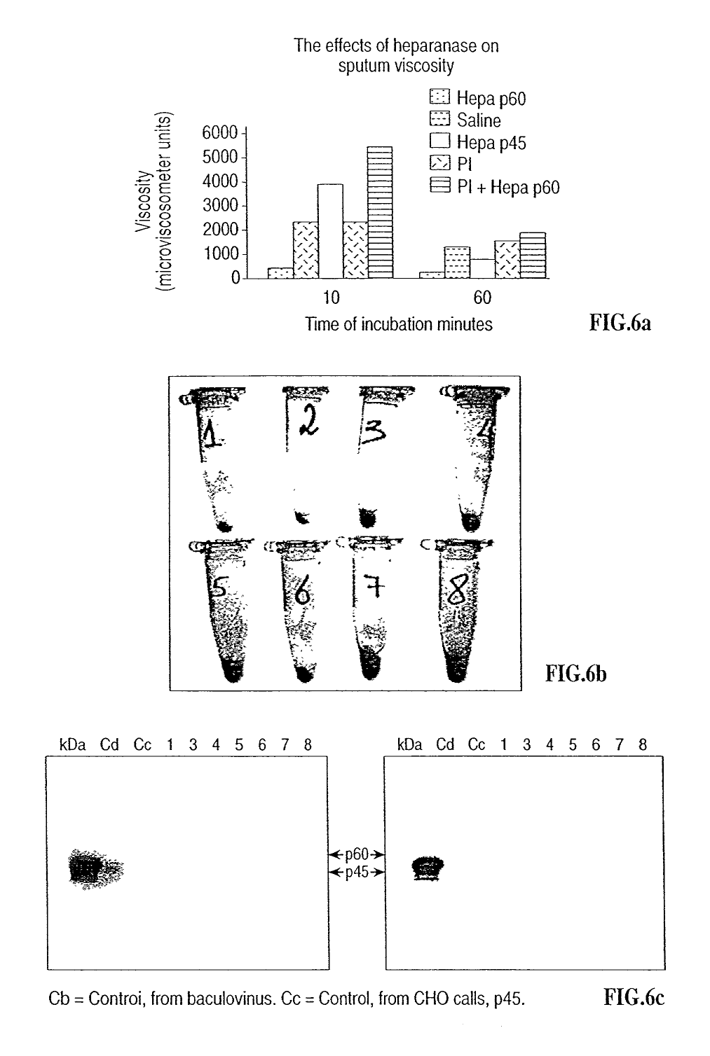

The adhered heparanase underwent proteolytic cleavage and activation. To show that heparanase was actually bound to the cells, the cells were subjected to Western blot analysis. It was found that not only that the enzyme was bound to the cells, but it was also processed from its inactive form, p60, to its active form, p45 (FIGS. 2b, 4c, 5b). These results indicate that the pro-enzyme may be a good drug for in vivo clinical treatment, and perhaps even better than the processed enzyme. Another evidence for the fact that the p60 heparanase undergoes proteolytic cleavage, and is therefore very active, comes from the liquefying effect of heparanase on sputum samples from cystic fibrosis patients (FIGS. 6a-b). It was found that p60 heparanase, when added to sputum samples, significantly reduced its viscosity within minutes. In contrast, when protease inhibitors were added to sputum samples prior to the addition of the enzyme, the enzyme did not reduce the viscosity of the sputum samples. The proteolytic cleavage of the enzyme by the sputum's innate proteases was confirmed by Western blot analysis (FIG. 6c). In this respect see also U.S. patent application Ser. No. 09/046,475, filed Mar. 25, 1998, which is incorporated herein by reference.

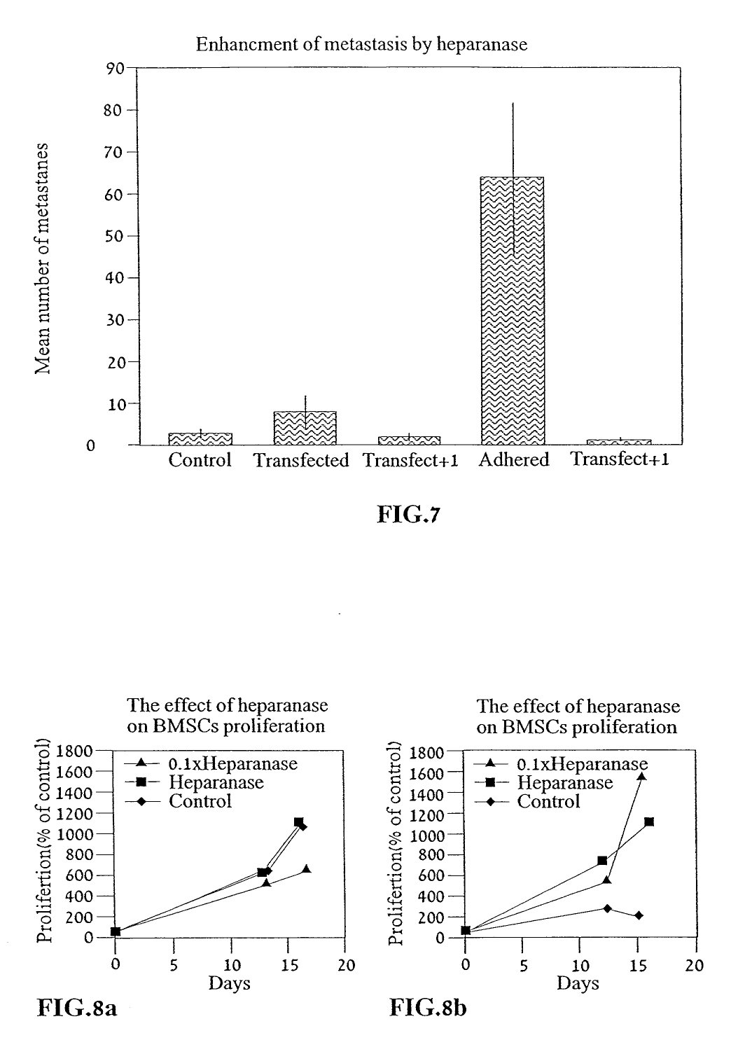

The adhered heparanase increases the metastatic potential of B16-F1 cells in vivo: In order to test the effect of adhered p60 heparanase on extravazation and invasiveness of cells, the enzyme was adhered to the low-metastatic B16-F1 cells, and the cells were injected to C57BL mice. After 3 weeks the animals were euthenized, the lungs were excised, and the number of metastases was counted. The results which are displayed in FIG. 7 show that the animals that were injected with the treated cells had 23 fold more metastases in the lungs, as compared to control animals which were injected with untreated cells, while animals that were injected with cells that were transfected with the heparanase cDNA had 3 fold more metastases as is compared to controls. Furthermore, when fragmin, which is known to inhibit heparanase, was injected concomitant with the treated cells, the number of metastases found in the lungs was markedly reduced to control levels.

The following section further describes the fate of the injected mice.

No abnormal clinical signs were detected in any of the animals during the entire study period. One animal from Group 4 (No. 19) was found dead in cage on day 4 of the study (three days following dosing).

The following Table presents mean body weight values (grams) and standard deviation (SD) of mice during the study period. Individual values are presented in Appendix.

| | Mean ± SD Body Weight (g) | | | | |
|---|---|---|---|---|---|
| Test Group | Day-1(*) | Day-9 | Day-13 | Day-18 | Day-21 |
| 1 | 23 ± 1.26 | 23.7 ± 1.21 | 24.9 ± 1.26 | 25.1 ± 1.34 | 24.8 ± 1.19 |
| (n = 6) | | | | | |
| 2 | 21.0 ± 1.87 | 23.1 ± 1.55 | 24.1 ± 1.63 | 24.9 ± 1.41 | 24.6 ± 1.39 |
| (n = 6) | | | | | |
| 3 | 22.1 ± 1.42 | 23.5 ± 1.92 | 24.1 ± 2.46 | 24.5 ± 2.67 | 24.8 ± 2.90 |
| (n = 6) | | | | | |
| 4 | 21.8 ± 3.13 | 22.7 ± 4.21 | 23.6 ± 4.51 | 24.3 ± 4.07 | 24.3 ± 4.67 |
| (n = 5) | | | | | |
| 5 | 21.4 ± 1.06 | 22.2 ± 1.82 | 23.2 ± 2.03 | 24.0 ± 1.93 | 24.4 ± 1.82 |
| (n = 7) | | | | | |
| 6 | 24.1(**) | 26.2 | 26.6 | 27.6 | 27.1 |
| (n = 2) | 19.7(**) | 22.2 | 24.0 | 24.9 | 25.2 |

(*)Body weight on the day of dosing;
(**)Since only two animals in this group, the actual values are presented, with no mean and SD.

The following Table presents metastases quantitative assessment at the time of study termination:

| | Animal | Metastases | | | | |
|---|---|---|---|---|---|---|
| Group No. | No. | Lungs | Liver | Thymus | Intestine | Heart |
| 1 | 1 | 4 | 0 | 0 | 0 | 0 |
| | 2 | 2 | 0 | 0 | 0 | 0 |
| | 3 | 2 | 0 | 0 | 0 | 0 |
| | 4 | 1 | 0 | 0 | 0 | 0 |
| | 5 | 2 | 0 | 0 | 0 | 0 |
| | 6 | 3 | 0 | 0 | 0 | 0 |
| Total per group | | 14 | 0 | 0 | 0 | 0 |
| 2 | 7 | 16 | 2 | 0 | 0 | 1 |
| | 8 | 1 | 1 | 0 | 0 | 0 |
| | 9 | 7 | 16 | 1 | 0 | 1 |
| | 11 | 4 | 0 | 0 | 0 | 0 |
| | 12 | 1 | 0 | 0 | 0 | 1 |
| | 31 | 13 | 1 | 0 | 0 | 0 |
| Total per group | | 42 | 20 | 1 | 0 | 3 |
| 3 | 13 | 3 | 0 | 0 | 0 | 0 |
| | 14 | 1 | 0 | 0 | 0 | 0 |
| | 15 | 0 | 0 | 0 | 0 | 0 |
| | 16 | 1 | 1 | 0 | 0 | 0 |
| | 17 | 0 | 0 | 0 | 1 | 0 |
| | 18 | 2 | 2 | 0 | 0 | 0 |
| Total per Group | | 7 | 3 | 0 | 1 | 0 |
| 4 | 20 | 132 | 0 | 0 | 0 | 0 |
| | 21 | 28 | 0 | 1 | 0 | 0 |
| | 22 | 64 | 0 | 0 | 0 | 0 |
| | 23 | 55 | 1 | 0 | 0 | 0 |
| | 24 | 43 | 0 | 0 | 0 | 0 |
| Total per Group | | 322 | 1 | 1 | 0 | 0 |
| 5 | 25 | 0 | 0 | 0 | 0 | 0 |
| | 26 | 1 | 0 | 0 | 0 | 0 |
| | 27 | 1 | 0 | 0 | 0 | 0 |
| | 28 | 2 | 0 | 0 | 0 | 0 |
| | 29 | 0 | 0 | 0 | 0 | 0 |
| | 30 | 0 | 0 | 0 | 0 | 0 |
| | 32 | 2 | 0 | 0 | 0 | 0 |
| Total per Group | | 6 | 0 | 0 | 0 | 0 |
| 6 | 33 | 0 | 0 | 0 | 0 | 0 |
| | 34 | 0 | 0 | 0 | 0 | 0 |
| Total per Group | | 0 | 0 | 0 | 0 | 0 |

These results suggest that heparanase catalyzes extravazation of cells, and other substances (e.g., drug delivery systems), through blood vessels, blood-brain-barrier, blood-milk barrier etc., and may ameliorate the invasion into the receiving tissues. This may result in the acceleration of the efficacy of implantation and transplantation, as well as enable cells, microorganisms and possibly other substances to cross biological blood barriers.

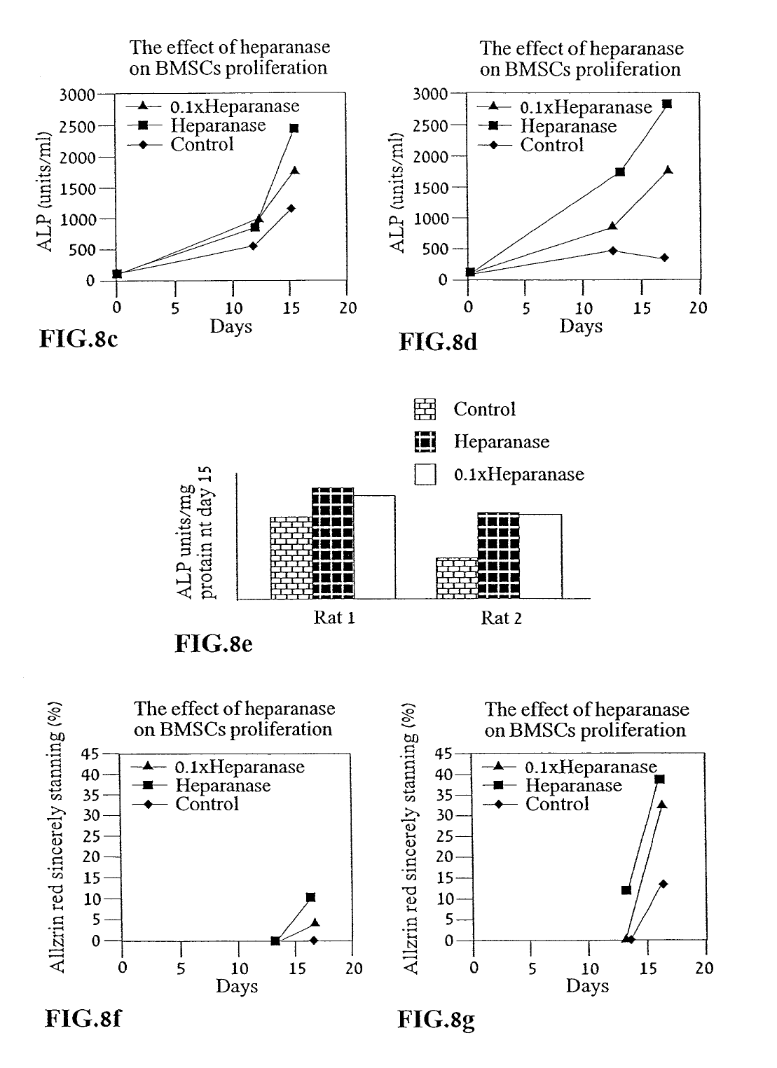

The effects of heparanase on bone formation: In order to test the effect of heparanase on tissue regeneration the effects of heparanase on bone formation were studied using stromal cells from the femoral bone marrow of young adult rats cultured for 15 days in the presence of beta-glycerolphosphate and dexamethasone. Stereoscopic microscope showed nodule formation after 14 days of culturing and both the number and the size of the nodules increased with time. The effect of heparanase on BMSCs proliferation was tested using the MTT proliferation test. The proliferation rate of treated cells was higher than that of non-treated cells (FIG. 8a-b). The effect of heparanase on BMSCs differentiation was tested by measuring the alkaline phosphatase (ALP) activity. The ALP activity was 2-4 fold higher in the treated cells after 15 days (FIG. 8c-d). The relative ALP activity as compared to the total protein was also calculated (FIG. 8e) and was shown to be higher in the heparanase treated cells. Calcified nodule formation of treated cultures was measured by alizarin-red staining. The average area of stained nodules in the treated cells was 2.5-3 fold larger than that in the control cell cultures after 15 days (FIG. 8f-g).

These findings show that heparanase increases cell proliferation, stimulates differentiation and bone-like tissue formation in the rat bone marrow stromal cell cultures.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

What is claimed is:

1. A method of inducing bone formation in a subject in need thereof, the method comprising administering to the subject a therapeutically effective amount of heparanase.

2. The method of claim 1, wherein said heparanase is a proheparanase.

3. The method of claim 1, wherein said heparanase is a processed heparanase.

4. The method of claim 1, wherein the subject has a medical condition characterized by bone loss, reduced bone mass or bone fracture.

5. The method of claim 1, wherein the subject has osteogenesis imperfecta.

6. The method of claim 1, wherein the subject has osteoporosis.

7. The method of claim 1, wherein the subject has a periodontal disease or a mucopolysaccharidoses associated bone defect.

8. The method of claim 1, wherein the subject has a bone void.

9. The method of claim 1, wherein administering is via intrabone injection.

10. A pharmaceutical composition for inducing bone formation in a subject in need thereof, the pharmaceutical composition comprising as an active ingredient a therapeutically effective amount of heparanase, and a pharmaceutically acceptable carrier.

11. The pharmaceutical composition of claim 10, wherein said heparanase is a proheparanase.

12. The pharmaceutical composition of claim 10, wherein said heparanase is a processed heparanase.

13. The pharmaceutical composition of claim 10, contained in a container and identified in print for use in a subject having a medical condition characterized by bone loss, reduced bone mass or bone fracture.

14. The pharmaceutical composition of claim 10, contained in a container and identified in print for use in a subject having osteogenesis imperfecta.

15. The pharmaceutical composition of claim 10, contained in a container and identified in print for use in a subject having osteoporosis.

16. The pharmaceutical composition of claim 10, contained in a container and identified in print for use in a subject having a periodontal disease or a mucopolysaccharidoses associated bone defect.

17. The pharmaceutical composition of claim 10, contained in a container and identified in print for use in a subject having a bone void.

18. The pharmaceutical composition of claim 10, formulated for administration via intrabone injection.
